(19) United States
(12) Patent Application Publication
(10) Pub. No.: US 2021/0290934 A1
(43) Pub. Date: Sep. 23, 2021

(54) INDWELLING HYPER-DIMENSIONAL CARDIAC PHYSIOLOGIC DATA LOGGING AND TRANSMISSION SYSTEM AND METHOD OF DOING BUSINESS

(71) Applicant: VIADERM LLC, Plymouth, MI (US)

(72) Inventor: Allen B. Kantrowitz, Miami Beach, FL (US)

(73) Assignee: VIADERM LLC, Plymouth, MI (US)

(21) Appl. No.: 17/260,267

(22) PCT Filed: Jul. 17, 2019
(86) PCT No.: PCT/US2019/042273

§ 371 Date: Jan. 14, 2021

Related U.S. Application Data
(60) Provisional application No. 62/699,301, filed on Jul. 17, 2018.

(51) Int. Cl. A61M 60/148; A61M 60/562; G06N 20/00; A61B 5/0205; A61B 5/00; A61B 5/07

(52) U.S. Cl. CPC A61M 60/148; A61M 60/562; G06N 20/00; A61M 2205/3303; A61B 5/4836; A61B 5/076; A61B 5/6869; A61B 5/0205

(57) ABSTRACT

A system to optimize the operation of counter pulsatile cardiac assist devices includes a collection of intracorporeal and extracorporeal physiologic sensors that generate data. A control system in electrical communication with the collection of intracorporeal and extracorporeal physiologic sensors generates control signals for the counter pulsatile cardiac assist device based on a counter pulsating aortic pumping element based on the data. A low power transmitter is in electrical communication with the collection of sensors and the control system and sends the generated data and the control signals to an external computer for aggregation and analysis. The analysis is based on a set of inputs from an implanted counter pulsatile cardiac assist devices.

### FIELD OF THE INVENTION

The present invention in general relates to the field of physiologic data monitoring, and more specifically to a system and method for capturing multiple correlated streams of physiologic and pharmacologic information on a continuous real-time basis, and securely uploading the data to a centralized computer system for further analysis.

### BACKGROUND OF THE INVENTION

The human race is experiencing an explosion of information. In 2018, every two days as much data is created as all the data generated from the beginning of time until 2000, and the amount of data being created continues to increase rapidly; by 2020, the amount of digital information available will have grown from around 5 zettabytes today to 50 zettabytes.

Today, almost every action taken leaves a digital trail or footprint. Data is generated whenever people go online, by global positioning systems (GPS), and GPS equipped smartphones, when people communicate with their friends through social media or chat applications, and when people shop. Furthermore, the amount of machine-generated data is also growing rapidly. Data is generated and shared when “smart” home devices communicate with each other or with their home servers. Industrial machinery in plants and factories around the world are increasingly equipped with sensors that gather and transmit data. In addition, medical devices and activity monitoring equipment generate vast amounts of data. The term “big data” refers to the collection of all this data and the advantageous use of the collected data across a wide range of areas, including business.

Big Data works on the principle that the more that is known about anything or any situation, the more reliably new insights and predictions are made about what will happen in the future. By comparing more data points, relationships begin to emerge that were previously hidden, and these relationships enable smarter decisions to be made. Most commonly, this is done through a process that involves building models, based on the data collected, and then running simulations, tweaking the value of data points each time and monitoring how the tweaks impact the results. This process may be automated with advanced analytics technology that runs millions of these simulations, tweaking all the possible variables until a pattern or an insight is found that helps solve the problem the process is working on.

Until relatively recently, collected data was limited to spreadsheets or databases that are very ordered and neat. Anything that wasn't easily organized into rows and columns was simply too difficult to work with and was ignored. Now though, advances in storage and analytics many different types of data may be captured, stored, and worked with. As a result, “data” can now mean anything from databases to photos, videos, sound recordings, written text, and sensor data.

In order to decipher of all of the varied data forms, big data projects often use advanced analytics involving artificial intelligence and machine learning. By teaching computers to identify what this data represents, including image recognition or natural language processing, the analysis systems can learn to spot patterns much more quickly and reliably than humans.

The analysis of big data has allowed business across almost every industry to improve operational performance. Companies can now accurately predict what specific segments of customers will want to buy, and when, to an incredibly accurate degree. Big data is also helping companies run their operations in a much more efficient way.

The use of big data has also been used to improve healthcare through the analysis of vast numbers of medical records and images for patterns that can help spot disease early and develop new medicines. Furthermore, sensor data from medical devices has been analyzed to improve the performance and effectiveness of medical devices.

Indwelling physiologic monitoring devices are a category of implanted medical devices which acquire important physiologic data and transmit the data to appropriate receiving equipment outside the body for further purposes. Examples of prior art implanted monitoring systems include: implanted cardiac loop recorders, implanted cardiac pacemakers and cardiac defibrillators, implanted glucose sensors, implanted posture/activity sensors for pain stimulators, and implanted long-term pressure transducers and flow transducers.

The implanted and indwelling physiologic monitoring devices measure, record, and transmit physiologic data which subsequently becomes the basis for decision making. The time-course associated with the decision making may be in physiologic real-time so that autonomous equipment with control algorithms can automatically trigger changes in medical treatment. Examples of such rapid data acquisition and analysis include automatic cardiac defibrillators and implanted glucose sensors combined with insulin drug pumps. Alternatively, the time-course of data acquisition and analysis may not be in real-time. An example of a non-real-time case is an implanted cardiac rhythm recorder. Moreover, the physiologic data may serve both real-time and non-real-time medical decision making. An example of use of data for both real-time and non-real-time would be a cardiac pacemaker, in which the physiologic data serves both real-time automatic changes in pacemaker function as well as non-real-time changes in medical management of the patient.

An example of an implanted and indwelling physiologic monitoring device is disclosed in U.S. Pat. No. 7,513,864 entitled SYNCHRONIZATION SYSTEM BETWEEN AORTIC VALVE AND CARDIAC ASSIST DEVICE that issued on Apr. 7, 2009, and herein incorporated by reference in its entirety. As disclosed therein an acoustic sensor or a mechanical force sensor is placed in proximity to the aortic valve in order to accurately sense aortic valve closure, with the use of this signal as a timing input to a cardiac assist device such as a left ventricular assist device (LVAD). The acoustic sensor takes the form of a microphone, ultrasonic transducer or Doppler sensor, where the acoustic sensor is dimensioned to be surgically implantable. Mechanical sensors disclosed included an accelerometer suitable for implantation.

As shown in FIG. 1 a cardiac system 10 is positioned relative to a mammalian left ventricle LV and aorta A. The system 10 is depicted as attached to the aorta A along the descending portion thereof. The system 10 includes an inflatable chamber 12 in fluid communication with a pump 14 by way of a fluid conduit 16. It is appreciated that while the inflatable chamber 12 is depicted as being coupled to a wall W of the aorta A that intra-aortic and cuff-type inflatable chambers are equally as well operative herewith. Additionally, while the inflatable chamber 12 is depicted as being located within the descending portion of the aorta A, placement within an ascending portion of an inflatable chamber within the ascending portion of the aorta A is also operative herewith. The relative location of an inflatable chamber relative to portions of the aorta being within the purview of one skilled in the art and including factors such as chamber size, chamber curvature, chamber elasticity, aorta curvature, and the type of inflatable chamber, as detailed above.

A sensor 18 responsive to acceleration forces or acoustic signatures associated with movement of the aortic valve AV is placed in proximity to the aortic valve AV. Preferably, the sensor 18 is placed within the fluid conduit 16 proximal to the inflatable chamber 12. The fluid conduit 16 transmits fluid between the pump 14 and inflatable chamber 12 to selectably inflate and deflate the chamber 12. Fluids used for this purpose illustratively include air, helium, nitrogen, argon, saline, and combinations thereof. The sensor 18 in this position is able to sense aortic valve AV movement from within the protective environment of the cardiac assist device and as such exhibits superior operating lifetime and offers greater ease of replacement, as compared to a conventional arterial pressure transducer. It is appreciated that the sensor 18 is also readily placed within the inflatable chamber 12 provided that care is taken so as not to damage the inflatable wall of the chamber 12 through contact with the sensor 18. It is further appreciated that other cardiac assist mechanisms are known to the art that are not based on driving a master slave compressor with compressable fluids, such as myoplasty, electromagnetic cuffs, and an cardiomyoplasty or aortomyplasty. Cardiomyoplasty and aortomyoplasty are well known to the art as evidenced by Hayward, Heart 1999; 82: 263-264; Salmons et al., Br. Heart J 1992; 68: 333-338; Lee et al., J. Thorac. Cardiovasc. Surg. 1991; 102(5): 757-765; and Girsch et al., Eur. J. Cardiothorac. Surg. 1998; 13(1): 78-83. The cardiac system 10 is equally operative herewith. The sensor 18 is a microphone, ultrasonic probe/transducer, Doppler sensor, or a mechanical force accelerometer. An exemplary implantable accelerometer operative herein is detailed in U.S. Pat. Nos. 3,972,038 and 5,674,258. Sensor signals whether collected in digital or analog form are in the form of a digital input signal to the timing controller 20 for the pump 14.

It is appreciated that in addition to monitoring the aortic valve AV closure event, additional events surrounding valve closure are also monitored. For instance, the ejection velocity of blood from the left ventricle LV slows and even reverses direction in some congenital conditions as the pressure on either side of the aortic valve AV equilibrates. A Doppler sensor is recognized to be able to measure velocity changes preceding the actual closure of the aortic valve AV. Further, subsequent to the closure of the aortic valve a back pressure is created thereon associated with the recoil of the aortic wall W. The mechanical systole associated with aortic wall recoil optionally serves as an additional parameter to precisely time the operation of a cardiac assist device by taking into consideration aortic elasticity to calculate with greater accuracy the specific instance of optimal assist device counter pulsation.

A sensor input signal to the pump controller 20 is used in a variety of ways to time chamber counter pulsation. By way of example, the historical timing of aortic valve closure from at least one preceding cardiac cycle is used to time cardiac assist device chamber inflation. Preferably, a windowing algorithm as described in U.S. Pat. No. 4,809,681 is used when historical cardiac cycle data is the signal input for inflatable chamber counter-pulsation. Alternatively, real-time sensor output is used to trigger chamber counter-pulsation for the same cardiac cycle. It is further appreciated that a combination of historical cardiac cycle and real-time cardiac cycle sensing are also operative to trigger assist device counter-pulsation.

A further prior art system of an implanted physiologic data source is CardioMEMS™. CardioMEMS™ are interogatable via an external electromagnetically coupled receiver, producing data to support a dynamic cardiovascular medical management system and motivate adjustment in medications as disclosed in WO 2014179739A1. The CardioMEMS™ pressure sensor is also described by U.S. Pat. No. 7,699,059 entitled “Implantable Wireless Sensor,” 7,679,355 entitled “Communicating with an Implanted Wireless Sensor,” and commonly assigned U.S. patent application Ser. Nos. 12/349,606, 12/175,803, 11/717,967, 11/613,645, 11/472,905, 11/276,571, 11/157,375, 11/105,294, and 10/943,772, which are incorporated in their entirety by reference herein. The CardioMEMS™ pressure sensors can be implanted in the pulmonary artery, more particularly in the distal pulmonary artery branch, with a right heart catherization or as part of a graft, such as a AAA stent-graft, and are configured to be energized with radio frequency (RF) energy to return high-frequency, high-fidelity dynamic pressure information from a precisely-selected location within a patient's body.

While there are a large number of implanted physiologic data sources there continues to be a need for improved collection, aggregation, and analysis of sensor data streams to optimize the effective operation of counter pulsatile cardiac assist devices.

### BRIEF DESCRIPTION OF THE DRAWINGS

The present invention is further detailed with respect to the following drawings that are intended to show certain aspects of the present of invention, but should not be construed as limit on the practice of the invention, where like numbers have the same meaning in the different drawing views, and wherein:

FIG. 1 is a prior art schematic diagram of a synchronization system for controlling pulsation of a left ventricular assist device (LVAD); top view of an embodiment of a robot operative in embodiments of the present invention;

FIG. 2 is a schematic diagram of the system of FIG. 1 with the addition of a low power transmitter to provide sensor measurements to external communication devices or networked computing devices in accordance with embodiments of the invention; and

FIG. 3 is a schematic diagram illustrating an overall view of communication devices, computing devices, and mediums for implementing embodiments of the invention.

### DETAILED DESCRIPTION OF THE INVENTION

The present invention has utility as a system and method that captures multiple correlated streams of physiologic and pharmacologic information on a continuous real-time basis, and then reliably and securely uploads the data to a centralized computer system for further analysis. In a specific inventive embodiment multiple correlated streams of physiologic data from implanted sensors are used to optimize the effective operation of counter pulsatile cardiac assist devices.

In inventive embodiments real-time and non-real-time physiologic data sources are utilized to obtain data to be analyzed. In particular, a collection of intracorporeal and extracorporeal physiologic sensors that are needed for real-time functioning of a control system of a cardiac assist device based on a counter pulsating aortic pumping element, optionally supplemented with additional physiologic sensors, are used to obtain analysis data. The optional supplemental or additional physiologic sensors provide additional contemporaneous physiologic data illustratively including: posture sensors, patient physical activity sensors, cardiac wall motion sensors, biochemical sensors capable of detecting concentration of physiologically meaningful chemicals, both naturally occurring and pharmacologically administered, and additional data such as voice memos articulated in the immediate vicinity of the portable control console of the cardiac assist device.

In the inventive system, these multiple temporally correlated data streams, herein referred to as an “indwelling hyperdimensional cardiac physiologic data set”, may be gathered while the individual patient is non-ambulatory in a hospital setting and also gathered while the patient in fully ambulatory and active in a non-hospital setting. Consequently, this “indwelling hyperdimensional cardiac physiologic data set” provides an unprecedented opportunity to obtain real-time physiologic data in an ambulatory patient to further elucidate the myriad interactions between the patient's cardiovascular homeostatic mechanisms and the introduced forms of treatment including the aforementioned cardiac assist device system, contemporaneous pharmacologic interventions and additional clinical interventions. The “indwelling hyperdimensional cardiac physiologic data set” is reliably and confidentially transmitted to a central location for support of further “big data” and “artificial intelligence” storage and analysis. With appropriate safeguards for patient confidentiality, the “indwelling hyperdimensional cardiac physiologic data set” may be used to support business methods in which a company acquiring and storing the “indwelling hyperdimensional cardiac physiologic data set” could make the data set available to parties interested in the development and effectiveness of treatments and devices for cardiovascular disease for a fee.

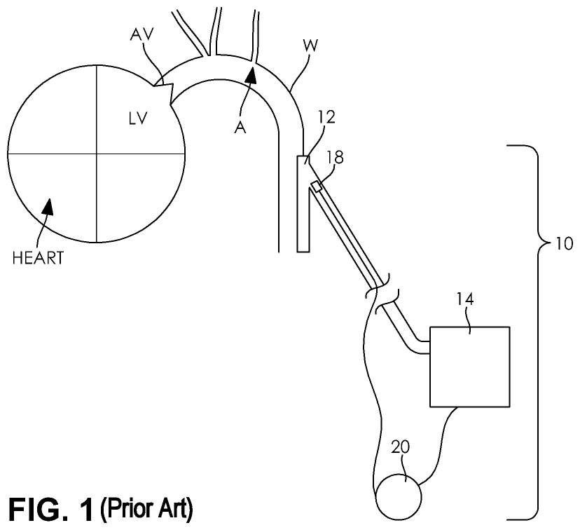

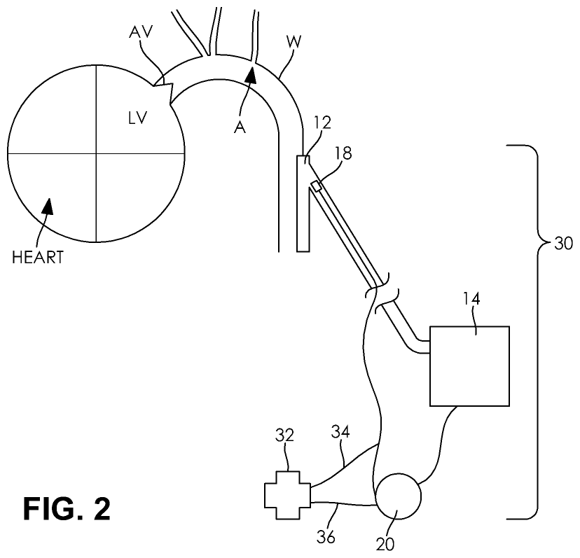

Referring now to the figures, FIG. 2 is a schematic diagram of a system 30 that differs from the system 10 of FIG. 1 with the addition of a low power transmitter 32 to provide sensor measurements and pulsation readings to external communication devices or networked computing devices within receiving distance of the low power transmitter 32. The low power transmitter 32 has wired electrical connections 34 to the sensor 18, and a wired connection 36 to the pump controller 20. A low power transmitter is used so as to not subject a patient to continuous higher power radio signals. The supplied sensor measurements and pulsation readings are then forwarded via the Internet or cellular networks by the communication devices including smartphones and tablets or networked computers to a central server where storage is provided for the supplied data and data analysis is performed.

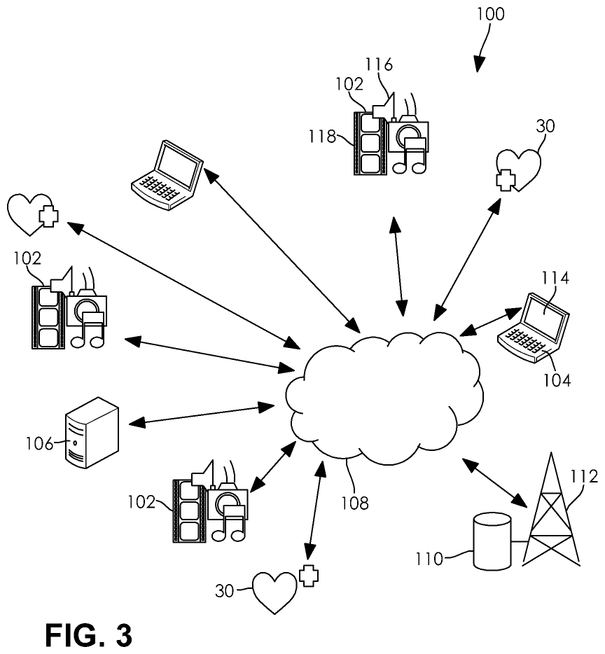

FIG. 3 is a schematic diagram illustrating an overall view of communication devices, computing devices, and mediums for implementing a system and method for monitoring and collecting data from a plurality of patients having counter pulsatile cardiac assist devices with data linking devices 30.

The system 100 includes multimedia devices 102 and desktop computer devices 104 configured with display capabilities 114 and processors for executing instructions and commands. The multimedia devices 102 are optionally mobile communication and entertainment devices, such as cellular phones, tablets, and mobile computing devices that in certain embodiments are wirelessly connected to a network 108. The multimedia devices 102 typically have video displays 118 and audio outputs 116. The multimedia devices 102 and desktop computer devices 104 are optionally configured with internal storage, software, and a graphical user interface (GUI) for obtaining and analyzing the collected data from the counter pulsatile cardiac assist devices with data linking devices 30 according to embodiments of the invention. A processor or group of processors may be used to execute the analysis of obtained data and implement machine learning and artificial intelligence (AI). The network 108 is optionally any type of known network including a fixed wire line network, cable and fiber optics, over the air broadcasts, local area network (LAN), wide area network (WAN), global network (e.g., Internet), intranet, etc. with data/Internet capabilities as represented by server 106. Communication aspects of the network are represented by cellular base station 110 and antenna 112. In a preferred embodiment, the network 108 is a LAN and each remote device 102 and desktop device 104 executes a user interface application (e.g., Web browser) to contact the server system 106 through the network 108. Alternatively, the remote devices 102 and 104 may be implemented using a device programmed primarily for accessing network 108 such as a remote client.

The software for the data acquisition and analysis, of embodiments of the invention, may be resident on tablets, 102 desktop or laptop computers 104, or stored within the server 106 or cellular base station 110 for download to an end user. Server 106 may implement a cloud-based service for implementing embodiments of the platform with a multi-tenant database for storage of separate client data for each separate medical device manufacturer or research group on the platform.

The foregoing description is illustrative of particular inventive embodiments of the invention, but is not meant to be a limitation upon the practice thereof. The following claims, including all equivalents thereof, are intended to define the scope of the invention.

1. A system to optimize the operation of counter pulsatile cardiac assist devices comprising:
a collection of intracorporeal and extracorporeal physiologic sensors that generate data;
a control system that generates control signals for the counter pulsatile cardiac assist device based on a counter pulsating aortic pumping element, the control system in electrical communication with the collection of intracorporeal and extracorporeal physiologic sensors; and
a low power transmitter in electrical communication with the collection of sensors and the control system, where the low power transmitter sends the generated data and the control signals to an external computer for aggregation and analysis, where the aggregation and analysis is based on a set of inputs from a plurality of implanted counter pulsatile cardiac assist devices.

2. The system of claim 1 further comprising additional physiologic sensors, the additional physiologic sensors comprising one or more of posture sensors, patient physical activity sensors, cardiac wall motion sensors, biochemical sensors capable of detecting concentration of physiologically meaningful chemicals, both naturally occurring and pharmacologically administered, and additional data comprising voice memos articulated in the immediate vicinity of a portable control console of the cardiac assist device.

3. The system of claim 1 further comprising communication devices including smartphones and tablets or networked computers within receiving distance of the low power transmitter; and
wherein the smartphones and the tablets or the networked computers are in communication with the external computer for aggregation and analysis.

4. The system of claim 1 wherein the analysis uses machine learning and artificial intelligence.

5. A method of using the system of claim 1 comprising:
collecting data from a plurality of implanted counter pulsatile cardiac assist devices; and
aggregating and analyzing the collected data.

6. The method of claim 5 wherein the aggregating and analyzing the collected data uses machine learning and artificial intelligence.
